(19) United States
(12) Patent Application Publication
(10) Pub. No.: US 2019/0350609 A1
(43) Pub. Date: Nov. 21, 2019

(54) ASPIRATION IRRIGATION DEVICE FOR LAPAROSCOPIC SURGERY

(71) Applicant: TELEFLEX MEDICAL INCORPORATED, Morrisville, NC (US)

(72) Inventors: Andrew SEROWSKI, Cary, NC (US); Salvatore CASTRO, Raleigh, NC (US)

(21) Appl. No.: 16/472,036

(22) PCT Filed: Dec. 20, 2017
(86) PCT No.: PCT/US17/67487

§ 371 Date: Jun. 20, 2019

Related U.S. Application Data
(60) Provisional application No. 62/438,878, filed on Dec. 23, 2016.

(51) Int. Cl. A61B 17/32; A61M 1/00

(52) U.S. Cl. CPC A61B 17/32002; A61M 1/0058; A61M 1/0035; A61M 1/0082; A61M 2205/106; A61B 2017/320028; A61B 2217/005; A61B 2217/007; A61M 2205/103; A61B 2017/320024

(57) ABSTRACT

A suction-irrigiation aspiration device may be provided with a tip capable of mechanical action to break up or emulsify material into smaller particles during a suction or irrigation procedure. The mechanical action in the tip may the in the form of a rotational or linear reciprocating motion to cut or emulsify the blood or tissue and thereby prevent or reduce clogging of the suction-irrigiation aspiration device.

### TECHNICAL FIELD

The present disclosure relates to a suction and irrigation device that is operable to apply suction force and/or apply a fluid for irrigation to a surgical site. The suction and irrigation device may be used to apply a fluid, such as an IV fluid, to the surgical site. The device may subsequently be used to clear the surgical site by providing a suction force to remove the IV fluid, blood, and/or tissue from the surgical site

### DESCRIPTION OF RELATED ART

In surgery, such as laparoscopic surgery, a small diameter suction tube may be used to apply a suction force to draw bodily fluids or secretions from a surgical site, and the same gibe may also be used to apply a fluid for irrigation to the surgical site. The suction and irrigation functions may be performed by attaching the small diameter suction tube to one end of a manifold that applies suction force and delivery of the irrigation fluid. The other end of the manifold may be attached to a suction generator and to a pressurized supply of irrigation fluid.

Where blood or soft tissue is present, the blood maycoagulate or the soft tissue may be of a size that will partially or completelyobstruct a suction tip or opening of the suction tube. When this occurs, the s on or operator may need to remove the suction tube from the surgical site, clean and unclog the suction tube, and then reinsert and direct the suction tube back to the surgical site before the surgical operation can continue. With each removal, the time needed to complete the surgery increases, which could lead to complications and increase cost. Accordingly, a need exists for a device that is capable of preventing or removing clogs during operation without having to remove the device from the surgical site.

### BRIEF DESCRIPTION OF THE DRAWINGS

FIG. 1 shows a perspective view of a rotary aspiration irrigation device in accordance with an aspect of the present disclosure.

FIG. 1A shows a close up view of an aspiration irrigation tip of the rotary aspiration irrigation device of FIG. 1.

FIG. 1B shows a close up, partial cross-sectional view of the aspiration irrigation tip of FIG. 1A.

FIG. 2 shows a side assembly view of the aspiration irrigation device of FIG. 1.

FIG. 3 shows a top view of the aspiration irrigation device of FIG. 1.

FIG. 4 shows a cross-sectional side view of the aspiration irrigation device of FIG. 3 taken at C-C.

FIG. 4A shows a close up, cross-sectional view of a rotary coupling of the aspiration irrigation device of FIG. 4

FIG. 5 shows a suction tip with a plurality of circular orifices for the rotary aspiration irrigation of FIG. 1 in accordance with an aspect of the present disclosure.

FIG. 6 shows a side cross-sectional view of the suction tip of FIG. 5.

FIG. 7 shows a close up, partial cross-sectional view of the suction tip of FIG. 5.

FIG. 8 shows a suction tip with a plurality of elongated orifices for the rotary aspiration irrigation of FIG. 1 in accordance with an aspect of the present disclosure.

FIG. 9 shows a side cross-sectional view of the suction tip of FIG. 8.

FIG. 10 shows a close up, partial cross-sectional view of the suction tip of FIG. 8 indicating at least a one mode of rotation in accordance with one aspect of the present disclosure.

FIG. 11 shows a close up, partial cross-sectional view of the suction tip of FIG. 8 indicating at least a one mode of rotation in accordance with another aspect of the present disclosure.

FIG. 12 shows a perspective view of a linear reciprocating aspiration irrigation device according with an aspect of the present disclosure.

FIG. 12A shows a close up view of the aspiration irrigation tip of the linear reciprocating aspiration irrigation device of FIG. 12.

FIG. 12B shows a close up, partial cross-sectional view of the linear reciprocating aspiration irrigation device of FIG. 12.

FIG. 12C shows a Varess needle introducer of the linear reciprocating aspiration irrigation device of FIG. 12 with the motor to valve coupling subassembly removed.

FIG. 12D a close up view of the Varess needle introducer of the linear reciprocating aspiration irrigation device of FIG. 12 with the motor to valve coupling subassembly removed.

FIG. 13 shows a side assembly view of the linear reciprocating aspiration irrigation device of FIG. 12.

FIG. 14 shows a top view of the linear reciprocating aspiration irrigation device of FIG. 12.

FIG. 15 shows a cross-sectional side view of the aspiration irrigation device of FIG. 14 taken at L-L.

FIG. 15A shows a close up view of a coupling mechanism for connecting and transmitting a reciprocating motion between a motor subassembly and a valve coupling assembly in accordance with an aspect of the present disclosure.

FIG. 16 shows a suction tip with a plurality of elongated circular slots the linear reciprocating aspiration irrigation device of FIG. 12 in accordance with an aspect of the present disclosure

FIG. 17 shows a side cross-sectional view of the suction tip of FIG. 16.

FIG. 18 shows a suction tip with a plurality of elongated circular slots the linear reciprocating aspiration irrigation device of FIG. 12 in accordance with an aspect of the present disclosure

FIG. 19 shows a side cross-sectional view of the suction tip of FIG. 16.

FIG. 20 shows a close up perspective, partial cross-sectional view of the suction tip of FIG. 16 in a first position.

FIG. 21 shows a close up perspective, partial cross-sectional view of the suction tip of FIG. 16 in a second position.

FIG. 22 shows a cross-sectional view of the Varess needle introducer of FIG. 12C.

FIG. 23 shows a close up view of an introducer needle from the Varess needle introducer shown in FIG. 22.

### DETAILED DESCRIPTION

Now referring to the drawings, wherein like reference numerals refer to like elements, exemplary aspects of the present disclosure will now be discussed.

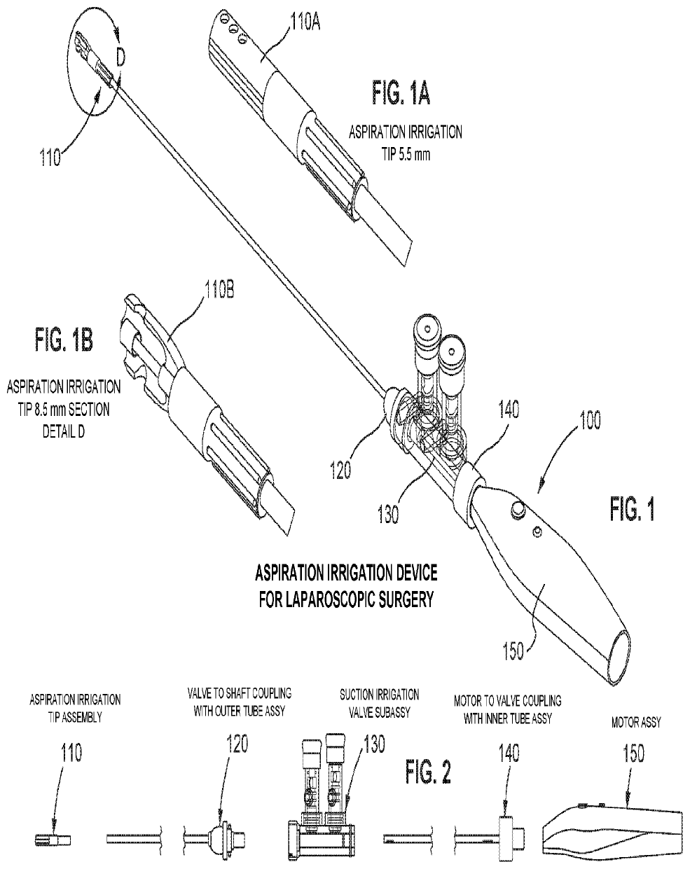

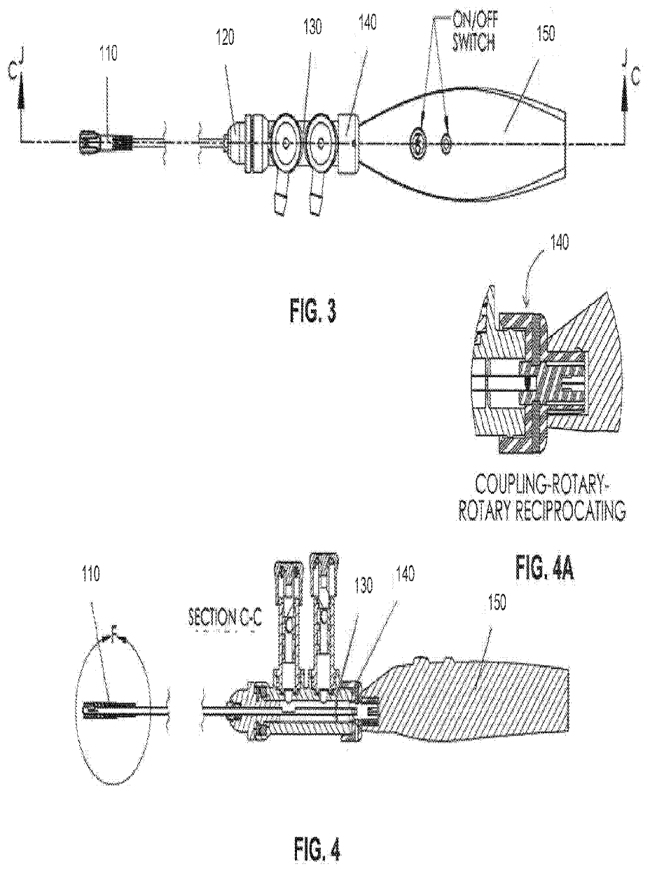

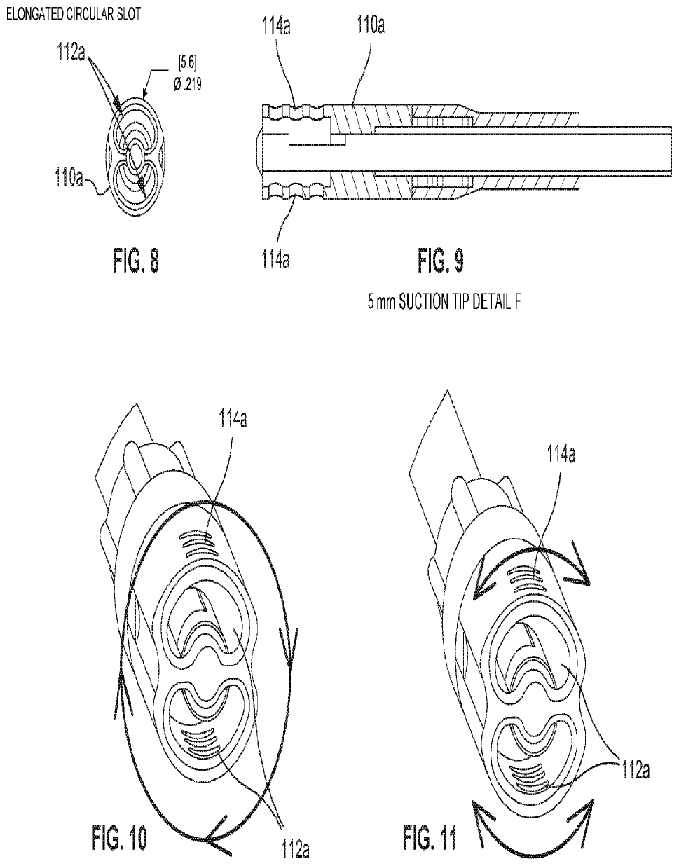

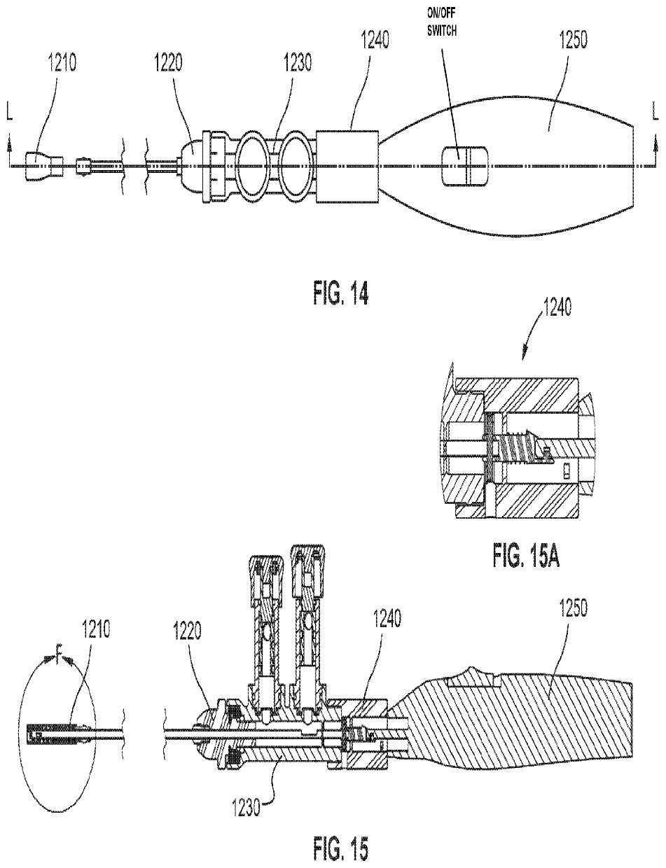

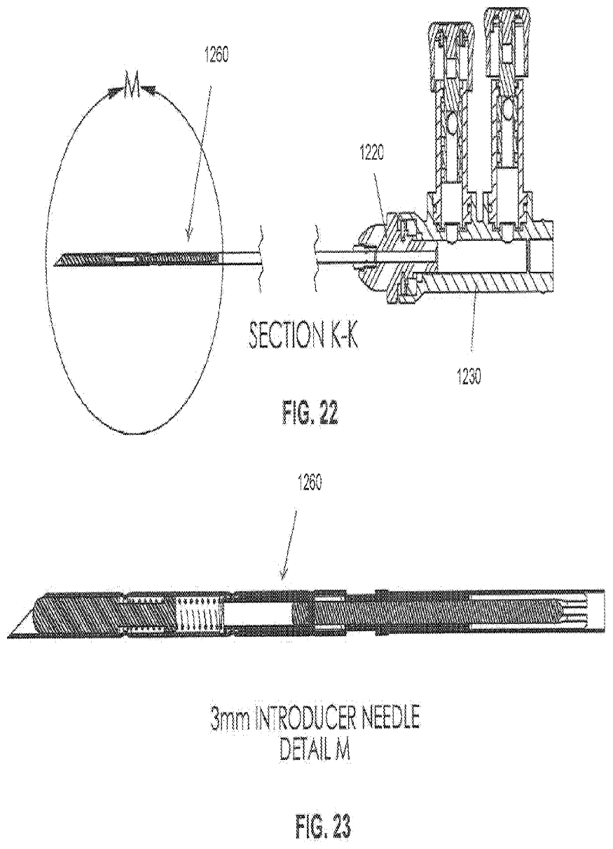

FIGS. 1-23 are generally directed towards suction and irrigation devices and their associated subassemblies and components which may be used to apply a fluid for irrigation and/or to apply a suction force during a surgical operation. During a surgical procedure, a tip of the surgical irrigation device may become obstructed with soft pieces of tissue and/or coagulated blood. In the related art, the tip of such a device must be removed from the surgical site in order for the tip and associate tube to be unclogged and clean before it can be reintroduced to the surgical site.

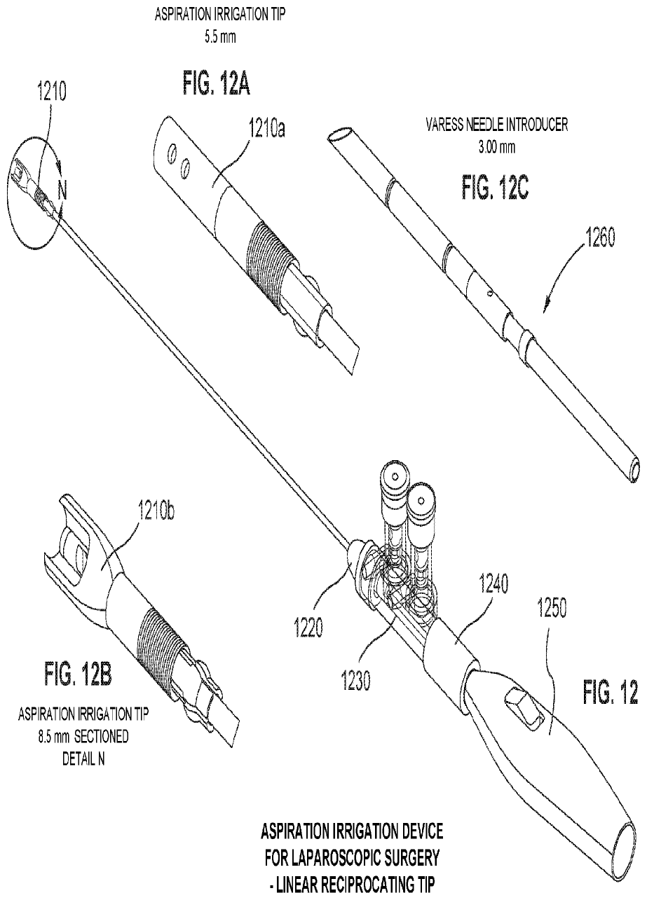

As will be discussed in further detail below, FIG. 1 generally shows an exemplary suction and irrigation device that may be used with a rotary or rotary reciprocating tip, and FIG. 12 generally shows an exemplary suction and irrigation device that may be used with a linear reciprocating tip. Both the rotary or rotary reciprocating tip and the linear reciprocating tip may be configured to reduce or prevent clogging when such tips are used to remove blood or soft pieces of tissue from a surgical site. It will be appreciated by one skilled in the art in view of the present disclosure that use of a rotary tip, a rotary reciprocating tip, and/or a linear reciprocating tip may be particularly useful in laparoscopic surgery and/or with suction and irrigation devices using tube diameters of 3 mm or less, in order to prevent clogging.

In accordance with an aspect of the present disclosure, clogging may be prevented by using the configured tip to cut or emulsify the blood or tissue entering against an opposing edge or surface. By cutting or emulsifying the material as it enters the tip of the suction and irrigation device, the material can be broken up into smaller particles such that it can more easily move through the tip and tube of the suction and irrigation device without clogging or jamming. Additionally, the suction and irrigation device may produce a vibration or ultrasonic motion at the tip to help further break up or emulsify material into smaller particles as it enters and passes through. Although three exemplary forms of mechanical action at the tip are described herein, it will be appreciated by one skilled in the art that minor variations, combinations, or alternative reciprocating motions may additionally or alternatively be applied to cut or emulsify blood or tissue to prevent clogs.

With reference to FIGS. 1-11, a suction-irrigation device with a rotary/rotary-reciprocating tip is shown. In operation, the rotary/rotary-reciprocating tip may be used to cut, slice and/or emulsify blood, tissue, and other organic debris. In accordance with an aspect of the present disclosure, as shown in FIGS. 1 and 2, a suction-irrigation device 100 with a rotary or rotary reciprocating tip 110a, 110b comprises multiple subassemblies. In one aspect, as shown in FIG. 2, the subassemblies may include a rotary or rotary reciprocating suction-irrigiation aspiration tip subassembly 110, a valve to shaft coupling with outer tube subassembly 120, a suction-irrigiation valve subassembly 130, a motor to valve coupling with inner tube subassembly 140, and a motor subassembly 150.

In one aspect, the valve to shaft coupling with outer tube subassembly 120 may be provided with a 3.0 mm outer shaft and a coupling which connects the suction-irrigiation valve assembly 130. The valve to shaft coupling with outer tube subassembly 120 may be used to interface and extend the functional tip through an abdomen wall, for example, to the suction-irrigiation valve subassembly 130. The valve to shaft coupling with outer tube subassembly 120 may also be used to encapsulate the inner tube that holds the suction-irrigation device 100 together.

In one aspect, the suction-irrigiation valve subassembly 130 may provide and control a suction force for removal of the discarded material from inside an abdominal surgical site, for example, and/or may provide an irrigation fluid to clean the interior of the abdominal surgical site. The suction force and or irrigation fluid may be applied to other surgical sites as would be appreciated by one skilled in the art in view of the present disclosure. In one aspect, the suction-irrigiation valve subassembly 130 may include a suction control valve, and the suction control valve may be interconnected with a tube attached to a suction source, such as a portable suction pump or a hospital suction system. In one aspect, the suction-irrigiation valve subassembly 130 may include an irrigation control valve, and the irrigation control valve may be interconnected with a tube attached to a fluid IV source or a pumping system. In one aspect, the suction-irrigiation valve subassembly 130 may be configured and purchased separately and may be customized to provide a more ergonomic and efficient control of suction and irrigation depending on the application and patient.

In one aspect, the motor to valve coupling with inner tube subassembly 140 may be provided to connect the tip of the inner tube to the opposite coupling and then to the motor subassembly 150. The inner tube is inserted through the suction-irrigiation valve into and through the valve to shaft coupling and the outer tube and then into the aspiration irrigation tip 110a, 110b. The inner tube in the tip may either rotate or reciprocate depending on the action provide by the motor subassembly 150. The inner tube may be open at the tip end and may have additional slot at the tip for the removal of material through the tube to the exit slots at the opposite end in the valve. The inner tube may be sealed while it is rotating at both couplings. The inner tube may be connected at the opposite end to a corresponding rotating coupling to translate the rotating or reciprocating action to and through the inner tube to its tip with the aspiration irrigation tip 110a, 110b.

In one aspect, the motor subassembly 150 may be provided to attach a motor to the valve coupling with inner tube subassembly 140. The motor subassembly 150 may be used to generation motion to move the inner tube and provide the action needed at the functioning tip 110a, 110b. In one aspect, three different motor assemblies may be provided to provide the motion needed for each of the methods to produce the different actions in the tips, the rotary, rotary-reciprocating and the linear-reciprocating actions. However, it is also contemplated that a single motor subassembly may be configured to selectively generate the motions necessary for use in each of the three methods.

In one aspect, the rotary or rotary reciprocating suction-irrigiation aspiration tip subassembly 110 may include an aspiration irrigation tip with an outer diameter of less than or equal to 8.5 mm. In accordance with another aspect, the rotary or rotary reciprocating suction-irrigiation aspiration tip subassembly 110 may include an aspiration irrigation tip 110a with an outer diameter of approximately 5.5 mm, as shown in FIG. 1A. In one aspect, the tip 110a may have an outer diameter of between 5.0 mm to 6.0 mm. In accordance with another aspect, the rotary or rotary reciprocating suction-irrigiation aspiration tip subassembly 110 may include an aspiration irrigation tip 110b with an outer diameter of approximately 8.5 mm, as shown in FIG. 1B. In one aspect, the tip 110b may have an outer diameter of between 8.0 mm to 9.0 mm.

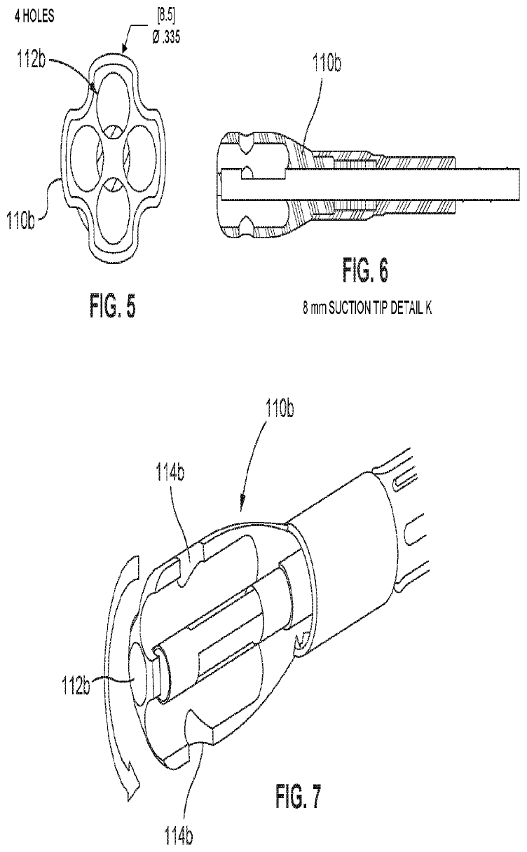

As shown in FIGS. 5-9, the aspiration irrigation tip 110a, 110b of the rotary or rotary reciprocating suction-irrigiation aspiration tip subassembly 110 may be provided with a plurality of openings 112a, 112b on a distal face of the tip. In one aspect, the rotary or rotary reciprocating suction-irrigiation aspiration tip 110a, 110b may be provided with two or more openings 112a, 112b on the distal face of the tip 110a, 110b. In accordance with one aspect, the distal face of the tip 110b may be provided with four openings 112b, and each of the openings may be defined by a circular slot or cutout. In accordance with one aspect, the distal face of the tip 110a may be provided with two openings 112ba, and each of the openings may be defined by an elongated circular or crescent-shaped slot or cutout. Other shapes for the slots or cutouts are of course contemplated and would be appreciated by one skilled in the art in view of the present disclosure. In accordance with another aspect, multiple shapes and/or sizes for the slots and cutouts may be applied to the same tip 110a, 110b. In one aspect, as shown in FIGS. 6, 7, 9, 10, and 11, a circumferential surface of the tip 110a, 110b may be provided with a plurality of lateral openings 114a, 114b. In one aspect, two or more lateral openings 114a, 114b may be provided on the circumferential surface of the tip 110a, 110b.

With specific reference to FIGS. 7 and 10, the tip 110a, 110b may be rotated continuously in one direction (clockwise or counter-clockwise) to cut and emulsify material as suction is being applied by the suction-irrigation device 100. In one aspect, a front end of the tube constantly rotates over a gap cutting the material in a circular sawing action as the suction force pulls the material against it. A slot, located further within the tube, rotates and cuts/emulsifies by scissoring/slicing the material between the side rotating edge of the tube slot and the stationary edges of the suction opening as the suction force pulls material into the slot and when cut into the tube.

With specific reference to FIGS. 7 and 11, the tip 110a, 110b may be rotated intermittently between a clockwise and counter-clockwise direction. Rather than rotating a complete 360 degrees as with the continuous rotational method described above, a front end of the tube rotates back and forth in one then the opposite direction. During this cycling between the two rotational directions, the tube edge moves in both direction to cut/emulsify material. Both sides of rotating edges of the slot cut/emulsify by scissoring/slicing the material against the both opposite stationary edges of the suction opening.

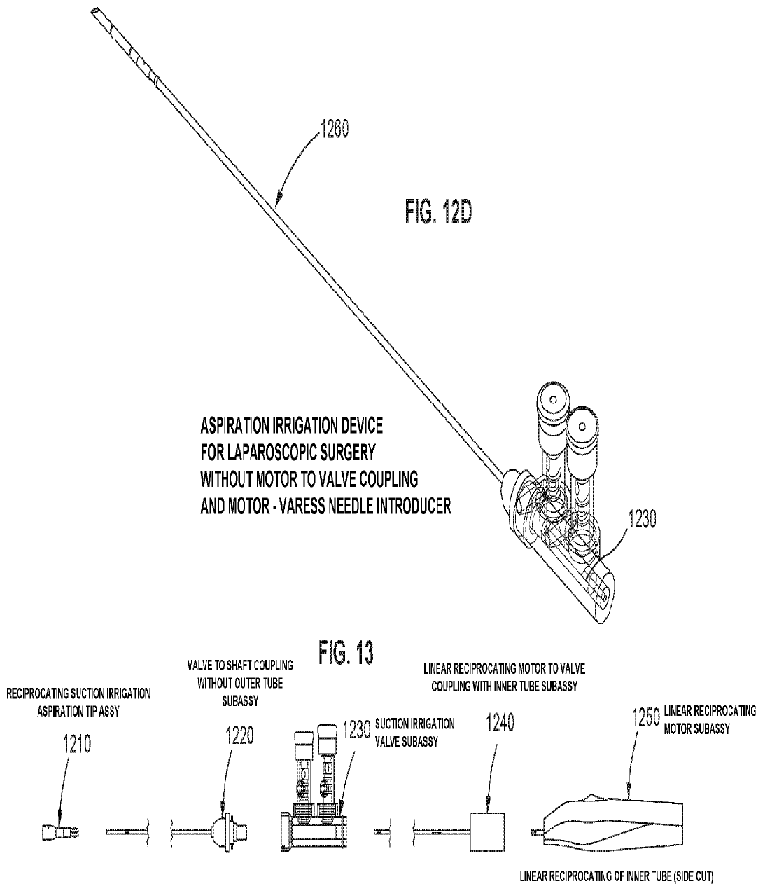

With reference to FIGS. 12-23, a suction-irrigiation device with a linear reciprocating tip is shown. In operation, the linear tip may be used to cut, slice and/or emulsify blood, tissue, and other organic debris. In accordance with an aspect of the present disclosure, as shown in FIGS. 12 and 13, a suction-irrigation device 1200 with a linear reciprocating tip 1210a, 1210b comprises multiple subassemblies. In one aspect, as shown in FIG. The subassemblies may include a linear reciprocating suction-irrigiation aspiration tip subassembly 1210, a valve to shaft coupling with outer tube subassembly 1220, a suction-irrigiation valve subassembly 1230, a motor to valve coupling with inner tube subassembly 1240, and a motor subassembly 1250. The suction-irrigation device 1200 may further comprise a Varess needle introducer 1260 as shown in FIGS. 12C and 12D. In accordance with one aspect, the Varess needle introducer 1260 attaches to the inside of the outer tube of the valve to shaft coupling. The Varess needle introducer 1260 may have have an outside diameter profile that is the same as the diameter of the tube. By matching the diameter, the Varess needle introducer 1260 and tube may be more easily inserted and slid into abdomen.

In one aspect, the valve to shaft coupling with outer tube subassembly 1220 may be provided with a 3.0 mm outer shaft and a coupling which connects the suction-irrigiation valve assembly 1230. The valve to shaft coupling with outer tube subassembly 1220 may be used to interface and extend the functional tip through an abdomen wall, for example, to the suction-irrigiation valve subassembly 1230. The valve to shaft coupling with outer tube subassembly 1220 may also be used to encapsulate the inner tube that holds the suction-irrigation device 1200 together.

In one aspect, the suction-irrigiation valve subassembly 1230 may provide and control a suction force for removal of the discarded material from inside an abdominal surgical site, for example, and/or may provide an irrigation fluid to clean the interior of the abdominal surgical site. The suction force and or irrigation fluid may be applied to other surgical sites as would be appreciated by one skilled in the art in view of the present disclosure. In one aspect, the suction-irrigiation valve subassembly 1230 may include a suction control valve, and the suction control valve may be interconnected with a tube attached to a suction source, such as a portable suction pump or a hospital suction system. In one aspect, the suction-irrigiation valve subassembly 1230 may include an irrigation control valve, and the irrigation control valve may be interconnected with a tube attached to a fluid IV source or a pumping system. In one aspect, the suction-irrigiation valve subassembly 1230 may be configured and purchased separately and may be customized to provide a more ergonomic and efficient control of suction and irrigation depending on the application and patient.

In one aspect, the motor to valve coupling with inner tube subassembly 1240 may be provided to connect the tip of the inner tube to the opposite coupling and then to the motor subassembly 1250. The inner tube is inserted through the suction-irrigiation valve into and through the valve to shaft coupling and the outer tube and then into the aspiration irrigation tip 1210a, 1210b. The inner tube in the tip may either rotate or reciprocate depending on the action provide by the motor subassembly 1250. The inner tube may be open at the tip end and may have additional slot at the tip for the removal of material through the tube to the exit slots at the opposite end in the valve. The inner tube may be sealed while it is rotating at both couplings. The inner tube may be connected at the opposite end to a corresponding rotating coupling to translate the rotating or reciprocating action to and through the inner tube to its tip with the aspiration irrigation tip 1210a, 1210b.

In one aspect, the motor subassembly 1250 may be provided to attach a motor to the valve coupling with inner tube subassembly 1240. The motor subassembly 1250 may be used to generation motion to move the inner tube and provide the action needed at the functioning tip 1210a, 1210b. In one aspect, three different motor assemblies may be provided to provide the motion needed for each of the methods to produce the different actions in the tips, the rotary, rotary-reciprocating and the linear-reciprocating actions. However, it is also contemplated that a single motor subassembly may be configured to selectively generate the motions necessary for use in each of the three methods.

In accordance with an aspect of the present disclosure, the linear reciprocating suction-irrigiation aspiration tip subassembly 1210 may include an aspiration irrigation tip with an outer diameter of less than or equal to 8.5 mm. In accordance with another aspect, the linear reciprocating suction-irrigiation aspiration tip subassembly 1210 may include an aspiration irrigation tip 1210a with an outer diameter of approximately 5.5 mm, as shown in FIG. 1A. In one aspect, the tip 1210a may have an outer diameter of between 5.0 mm to 6.0 mm. In accordance with another aspect, the linear reciprocating suction-irrigiation aspiration tip subassembly 1210 may include an aspiration irrigation tip 1210b with an outer diameter of approximately 8.5 mm, as shown in FIG. 1B. In one aspect, the tip 1210b may have an outer diameter of between 8.0 mm to 9.0 mm.

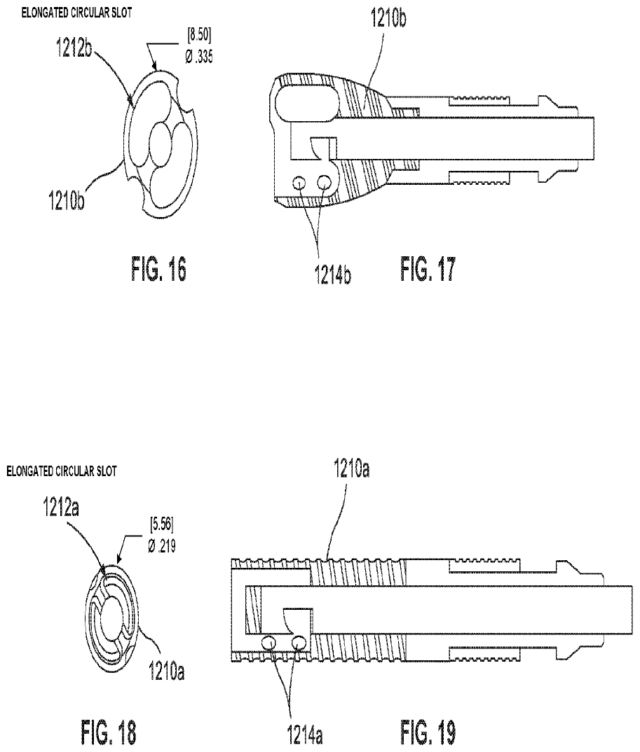

As shown in FIGS. 16-19, the aspiration irrigation tip 1210a, 1210b of the linear reciprocating suction-irrigiation aspiration tip subassembly 1210 may be provided with a plurality of openings 1212a, 1212b on a distal face of the tip 1210a, 1210b. In one aspect, the aspiration irrigation tip 1210a, 1210b may include two or more elongated circular or crescent-shaped slot or cutout, as shown generally in FIGS. 16 and 18. In one aspect, as shown in FIGS. 17 and 19, the aspiration irrigation tip 1210a, 1210b may be provided with a plurality of lateral openings 1214a, 1214b. In one aspect, two or more lateral openings 1214a, 1214b may be provided on the circumferential surface of the tip 1210a, 1210b. Other shapes for the slots or cutouts are of course contemplated and would be appreciated by one skilled in the art in view of the present disclosure. In accordance with another aspect, multiple shapes and/or sizes for the slots and cutouts may be applied to the same tip 1210a, 1210b.

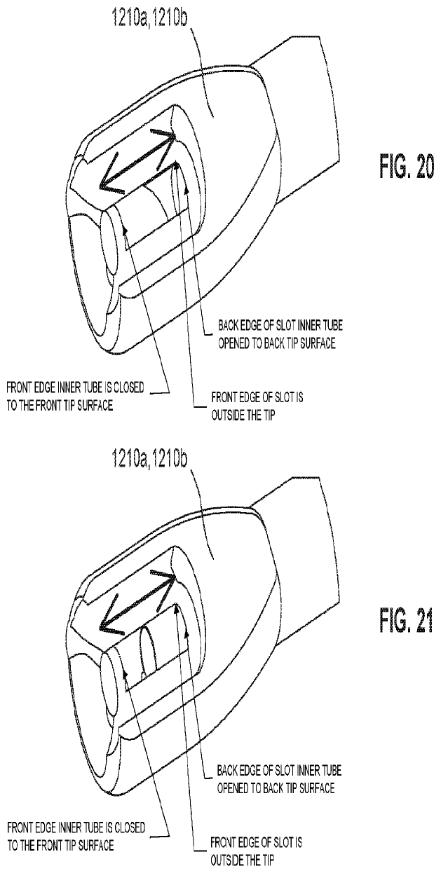

With specific reference to FIGS. 20 and 21, the tip 1210a, 1210b may be linearly actuated to move back and forth. front tube edge moves back and forth in both directions but linearly along the axis to cut/emulsify the material against the front surface. The slot tube edge moves back and forth against the back stationary edge scissoring/slicing the material as it is pulled in by suction applied by the suction-irrigation device 1200. In one aspect, the forward action allows the suction to be closed as it cuts/emulsifies the material at the front and opens the suction when it is not cutting with the slot in the back, and backward action reverses the suction action.

While the apparatus and methods of the present disclosure have been shown and described, it will be appreciated that the foregoing description provides examples tips for use with suction and irrigation devices. However, it is contemplated that other implementations of the disclosure may differ in detail from the foregoing examples. Each aspect of the disclosure may be used individually or in combination with one another, as will be understood by one skilled in the art in view of the present disclosure. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

1. An aspiration irrigation device comprising:
a reciprocating suction-irrigation aspiration tip subassembly;
a suction-irrigation valve subassembly; and
a motor subassembly.

2. The aspiration irrigation device of claim 1, further comprising a valve to shaft coupling with outer tube subassembly.

3. The aspiration irrigation device of claim 2, wherein the valve to shaft coupling with outer tube subassembly includes an outer shaft with a 3.0 mm diameter.

4. The aspiration irrigation device of claim 1, a motor to valve coupling with inner tube subassembly.

5. The aspiration irrigation device of claim 1, wherein the reciprocating suction-irrigation aspiration tip subassembly includes a rotary or rotary reciprocating tip.

6. The aspiration irrigation device of claim 5, wherein the rotary or rotary reciprocating tip has an outer diameter of less than or equal to 8.5 mm.

7. The aspiration irrigation device of claim 5, wherein the rotary or rotary reciprocating tip has an outer diameter of 5.0 mm to 6.0 mm.

8. The aspiration irrigation device of claim 5, wherein the rotary or rotary reciprocating tip is provided with two or more openings on a distal face of the rotary or rotary reciprocating tip.

9. The aspiration irrigation device of claim 8, wherein the two or more openings are defined by an elongated circular or crescent-shaped slot or cutout.

10. The aspiration irrigation device of claim 5, wherein the rotary or rotary reciprocating tip is configured to rotate intermittently between a clockwise and a counter-clockwise direction.

11. The aspiration irrigation device of claim 1, wherein the reciprocating suction-irrigation aspiration tip subassembly includes a linear reciprocating tip.

12. The aspiration irrigation device of claim 11, wherein the linear reciprocating tip has an outer diameter of 5.0 mm to 6.0 mm.

13. The aspiration irrigation device of claim 11, wherein the linear reciprocating tip is provided with two or more openings on a distal face of the linear reciprocating tip.

14. The aspiration irrigation device of claim 13, wherein the two or more openings are defined by an elongated circular or crescent-shaped slot or cutout.

15. The aspiration irrigation device of claim 11, wherein the linear reciprocating tip is provided with two or more lateral openings provided on a circumferential surface of linear reciprocating the tip.

16. The aspiration irrigation device of claim 1, wherein the suction-irrigation valve subassembly includes a suction control valve.

17. The aspiration irrigation device of claim 16, wherein the suction control valve is attached to a suction source via a tube.

18. The aspiration irrigation device of claim 17, wherein the suction source is a portable suction pump or a hospital suction system.

19. The aspiration irrigation device of claim 1, wherein the suction-irrigation valve subassembly includes an irrigation control valve.

20. The aspiration irrigation device of claim 19, wherein the irrigation control valve is attached to a fluid IV source or a pumping system.
